(19) United States
(12) Patent Application Publication
(10) Pub. No.: US 2023/0233127 A1
(43) Pub. Date: Jul. 27, 2023

(54) BIOSIGNAL SENSING ELECTRODE

(71) Applicant: Murata Manufacturing Co., Ltd., Nagaokakyo-shi (JP)

(72) Inventors: Kazuari SASAKI, Nagaokakyo-shi (JP); Takeshi TORITA, Nagaokakyo-shi (JP)

(21) Appl. No.: 18/127,249

(22) Filed: Mar. 28, 2023

Related U.S. Application Data
(63) Continuation of application No. PCT/JP2021/035414, filed on Sep. 27, 2021.

(30) Foreign Application Priority Data
Sep. 30, 2020 (JP) 2020-165302

(51) Int. Cl. A61B 5/263; H01B 5/14; A61B 5/268

(52) U.S. Cl. CPC A61B 5/263; H01B 5/14; A61B 5/268

(57) ABSTRACT

A biosignal sensing electrode that includes: a conductive film containing particles of a layered material including one or plural layers, the one or plural layers includes a layer body represented by: MₘXₙ, wherein M is at least one metal of Group 3, 4, 5, 6, or 7, X is a carbon atom, a nitrogen atom, or a combination thereof, n is 1 to 4, m is more than n and 5 or less, and a modifier or terminal T exists on a surface of the layer body, wherein T is at least one selected from a hydroxyl group, a fluorine atom, a chlorine atom, an oxygen atom, or a hydrogen atom; and a porous membrane that contains a hydrophilic polymer, the porous membrane having a first surface in contact with at least part of the conductive film and a second surface defining a contact surface with a subject.

### CROSS REFERENCE TO RELATED APPLICATIONS

The present application is a continuation of International application No. PCT/JP2021/035414, filed Sep. 27, 2021, which claims priority to Japanese Patent Application No. 2020-165302, filed Sep. 30, 2020, the entire contents of each of which are incorporated herein by reference.

### FIELD OF THE INVENTION

The present invention relates to a biosignal sensing electrode.

### BACKGROUND OF THE INVENTION

Examples of a method for detecting biological information such as an electrical signal from a muscle or a heart of a subject (patient) without inflicting pain or the like on a human body include a method for measuring the biological information by bringing a sheet-like electrode into contact with the subject. For example, Patent Document 1 discloses a biological electrode-coated pad using a hydrophilic gel containing water and an electrolyte. Patent Document 2 discloses a biopotential electrode including: (a) an electrical conductor; (b) a thin film selectively permeable to ion conduction to present a dry surface to the subject; and (c) a conductive medium disposed to communicate with a part of the electrical conductor and a part of the thin film. Meanwhile, in recent years, MXene has been attracting attention as a new material having conductivity. MXene is a type of so-called two-dimensional material, and as will be described later, is a layered material in the form of one or plural layers. In general, MXene is in the form of particles (which can include powders, flakes, nanosheets, and the like) of such a layered material. Patent Document 3 discloses a bioelectrode formed of a contact material containing MXene.

Patent Document 1: WO 2013/039151

Patent Document 2: U.S. Pat. No. 8,798,710

Patent Document 3: WO 2019/055784

### SUMMARY OF THE INVENTION

In the biological electrode-coated pad of Patent Document 1, when the moisture amount changes due to drying, the impedance changes, so that it is considered that it is difficult to obtain a highly accurate signal. In addition, since it contains water, it feels uncomfortable like being wet at the time of wearing. Since the biopotential electrode of Patent Document 2 has a large number of layers and a large number of layer interfaces, the impedance increases, and it is considered that it is difficult to obtain a highly accurate signal. Further, in Patent Document 3, MXene is used in a contact portion with a subject, but there is a possibility that MXene is desorbed by contact, and it is considered that it is difficult to stably measure over a long period of time.

An object of the present invention is to provide a biosignal sensing electrode which exhibits high conductivity (low impedance), suppresses peeling of a predetermined layered material (also referred to as “MXene” in the present specification), and does not cause discomfort at the time of wearing.

According to one aspect of the present invention, there is provided a biosignal sensing electrode comprising: a conductive film containing particles of a layered material including one or plural layers, wherein the one or plural layers includes a layer body represented by: MₘXₙ, wherein M is at least one metal of Group 3, 4, 5, 6, or 7, X is a carbon atom, a nitrogen atom, or a combination thereof, n is 1 to 4, m is more than n and 5 or less, and a modifier or terminal T exists on a surface of the layer body, wherein T is at least one selected from the group consisting of a hydroxyl group, a fluorine atom, a chlorine atom, an oxygen atom, or a hydrogen atom; and a porous membrane that contains a hydrophilic polymer, the porous membrane having a first surface in contact with at least part of the conductive film and a second surface defining a contact surface with a subject.

According to the present invention, there is provided a biosignal sensing electrode in which the biosignal sensing electrode includes a stacked layer of a conductive film containing particles of a predetermined layered material and a porous membrane, and the porous membrane is provided on a contact surface with a subject, whereby high conductivity (low impedance) is exhibited, peeling of MXene is suppressed, and discomfort is not caused at the time of wearing.

### BRIEF DESCRIPTION OF THE DRAWINGS

FIGS. 1(a) and 1(b) are diagrams illustrating a conductive film in one embodiment of a biosignal sensing electrode of the present invention, in which FIG. 1(a) illustrates a schematic cross-sectional view of the conductive film, and FIG. 1(b) illustrates a schematic perspective view of MXene in the conductive film.

FIGS. 2(a) and 2(b) are schematic cross-sectional views illustrating MXene which is a layered material usable for a conductive film of an electrode in one embodiment of the biosignal sensing electrode of the present embodiment, in which FIG. 2(a) illustrates single-layer MXene, and FIG. 2(b) illustrates multi-layered (exemplarily two-layered) MXene.

FIG. 3 is a schematic cross-sectional view illustrating a conductive film according to another embodiment of the present invention.

FIGS. 4(a) to 4(d) are diagrams exemplifying a pore shape of a porous membrane in the biosignal sensing electrode of the present invention.

FIG. 5 is a schematic cross-sectional view illustrating a biosignal sensing electrode according to one embodiment of the present invention.

FIG. 6 is a schematic cross-sectional view illustrating a biosignal sensing electrode according to another embodiment of the present invention.

FIG. 7 is a schematic perspective view illustrating a biosignal sensing electrode according to another embodiment of the present invention.

FIG. 8 is a schematic cross-sectional view illustrating a biosignal sensing electrode according to another embodiment of the present invention.

FIG. 9 is a schematic cross-sectional view illustrating a biosignal sensing electrode according to another embodiment of the present invention.

### DETAILED DESCRIPTION OF PREFERRED EMBODIMENTS OF THE INVENTION

Hereinafter, a biosignal sensing electrode in the embodiment of the present invention will be described in detail, but the present invention is not limited to such an embodiment.

The biosignal sensing electrode in the embodiment of the present invention includes a stacked layer of a conductive film containing particles of a layered material including one or plural layers and a porous membrane . First, each of the conductive film and the porous membrane will be described.

### Conductive Film

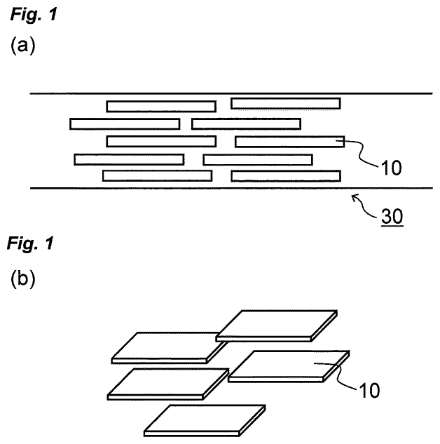

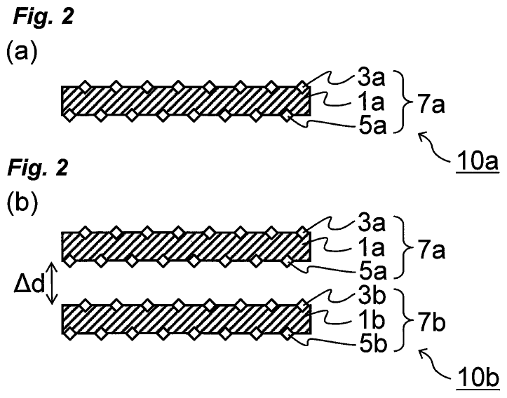

Referring to FIG. 1, a conductive film 30 included in the electrode of the present embodiment includes particles 10 of a predetermined layered material. The particles of a predetermined layered material included in the conductive film in the present embodiment are defined as follows.

A layered material including one or plural layers, wherein the layer includes a layer body represented by a formula below:

MₘXₙ

wherein M is at least one metal of Group 3, 4, 5, 6, or 7, and can comprise at least one selected from the group consisting of so-called early transition metals, for example, Sc, Ti, Zr, Hf, V, Nb, Ta, Cr, Mo, or Mn; X is a carbon atom, a nitrogen atom, or a combination thereof; n is not less than 1 and not more than 4; and m is more than n but not more than 5 (the layer body can have a crystal lattice in which each X is located in the octahedral array of M); and a modifier or terminal T exists on a surface of the layer body (more specifically, on at least one of two surfaces, facing each other, of the layered body), wherein T is at least one selected from the group consisting of a hydroxyl group, a fluorine atom, a chlorine atom, an oxygen atom, or a hydrogen atom (the layered material can be understood as a layered compound and also represented by “MₘXₙTₛ,” wherein s is any number and traditionally x may be used instead of s). Typically, n can be 1, 2, 3, or 4, but is not limited thereto.

In the above formula of Mxene, M is preferably at least one selected from the group consisting of Ti, Zr, Hf, V, Nb, Ta, Cr, Mo, or Mn, and more preferably at least one selected from the group consisting of Ti, V, Cr, or Mo.

Such Mxene can be synthesized by selectively etching (removing and optionally layer-separating) A atoms (and optionally parts of M atoms) from a MAX phase. The MAX phase is represented by the following formula:

MₘAXₙ

(wherein M, X, n, and m are as described above; and A is at least one element of Group 12, 13, 14, 15, or 16, is usually a Group A element, typically Group IIIA and Group IVA, more specifically, may include at least one selected from the group consisting of Al, Ga, In, Tl, Si, Ge, Sn, Pb, P, As, S, or Cd, and is preferably Al), and has a crystal structure in which a layer formed of A atoms is located between two layers (each X may have a crystal lattice located within an octahedral array of M) represented by MₘXₙ. Typically, in the case of m=n+1, the MAX phase has a repeating unit in which one layer of X atoms is disposed between the layers of M atoms of n+1 layers (these layers are also collectively referred to as “MₘXₙ layer”), and a layer of A atoms (“A atom layer”) is disposed as a next layer of the (n+1) th layer of M atoms; however, the present invention is not limited thereto. By selectively etching (removing and optionally layer-separating) A atoms (and optionally parts of M atoms) from the MAX phase, the A atom layer (and optionally parts of M atoms) is removed and, thus, the surface of the exposed MₘXₙ layer is modified by hydroxyl groups, fluorine atoms, chlorine atoms, oxygen atoms, hydrogen atoms, etc., existing in an etching liquid (usually, an aqueous solution of a fluorine-containing acid is used, but not limited thereto), so that the surface is terminated. The etching can be carried out using an etching liquid containing F⁻, and a method using, for example, a mixed liquid of hydrochloric acid and lithium fluoride which is also an intercalator, for both etching and intercalation, a method using hydrofluoric acid, or the like may be used. Then, the layer separation of Mxene (delamination, separating multilayer Mxene into single-layer Mxene) may be appropriately promoted by any suitable post-treatment (for example, intercalation using an intercalator as one of delamination treatment, ultrasonic treatment, handshake, automatic shaker, or the like). Since the shear force of an ultrasonic treatment is too large so that the Mxene can be destroyed, it is desirable to apply appropriate shear force by handshake, an automatic shaker or the like, when it is desired to obtain a two-dimensional Mxene (preferably single-layer Mxene) having a larger aspect ratio.

After the post-treatment, the supernatant containing the single-layer Mxene and/or about 2 to 5 layers of the few-layer Mxene and the subnatant containing the multilayer Mxene may be separated using a centrifugal separator. In the present embodiment, Mxene contained in the supernatant and/or the subnatant can be used as particles of the layered material. It is preferable to use Mxene contained in the supernatant containing the single-layer/few-layer Mxene as the layered material particles because low impedance is easily realized.

Mxenes whose above formula MₘXₙ is expressed as below are known:

Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti, V)₂C, (Ti,Nb)₂C, W₂C, W_{1.3}C, Mo₂N, Nb_{1.3}C, Mo_{1.3}Y_{0.6}C (in the above formula, “1.3” and “0.6” mean about 1.3 (= 4/3) and about 0.6 (=⅔), respectively),

Ti₃C₂, Ti₃N₂, Ti₃(CN), Zr₃C₂, (Ti,V)₃C₂, (Ti₂Nb)C₂, (Tᵢ₂Ta)C2, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C2, (Mo₂Zr)C2, (Mo₂Hf)C2, (Mo₂V)C2, (Mo₂Nb)C2, (Mo₂Ta)C2, (W₂Ti)C2, (W₂Zr)C2, (W₂Hf)C₂,

Ti₄N₃, V₄C₃, Nb₄C₃, Ta₄C₃, (Ti,Nb)₄C₃, (Nb,Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, (W₂Hf₂)C₃, (Mo_{2.7}V_{1.3})C₃ (in the above formula, “2.7” and “1.3” mean about 2.7 (= 8/3) and about 1.3 (= 4/3), respectively.)

Typically in the above formula, M can be titanium or vanadium and X can be a carbon atom or a nitrogen atom. For example, the MAX phase is Ti₃AlC₂ and Mxene is Ti₃C₂Tₛ (in other words, M is Ti, X is C, n is 2, and m is 3).

It is noted that in the present invention, Mxene may contain remaining A atoms at a relatively small amount, for example, at 10 mass % or less with respect to the original amount of A atoms. The remaining amount of A atoms can be preferably 8 mass % or less, and more preferably 6 mass % or less. However, even if the residual amount of A atoms exceeds 10 mass %, there may be no problem depending on the application and use conditions of conductive films.

As schematically illustrated in FIGS. 2(a) and 2(b), the Mxene (particles) 10 synthesized in this way can be a layered material containing one or plural Mxene layers 7a, 7b (as examples of the Mxene (particles) 10, FIG. 2(a) illustrates Mxene 10a of one layer, and FIG. 2(b) illustrates Mxene 10b of two layers, but is not limited to these examples). More specifically, the Mxene layers 7a, 7b have layer bodies (MₘXₙ layers) 1a, 1b represented by MₘXₙ, and modifiers or terminals T 3a, 5a, 3b, 5b existing on the surfaces of the layer bodies 1a, 1b (more specifically, on at least one of two surfaces, facing each other, of each layer). Therefore, the Mxene layers 7a, 7b are also represented by “MₘXₙTₛ,” wherein s is any number. Mxene 10 may be: one that exists as one layer obtained by such Mxene layers being separated individually (single-layer structure illustrated in FIG. 2(a), so-called single-layer Mxene 10a); a laminate made of a plurality of Mxene layers being stacked to be apart from each other (multilayer structure illustrated in FIG. 2(b), so-called multilayer Mxene 10b); or a mixture thereof. Mxene 10 can be particles (which can also be referred to as powders or flakes) as a collective entity composed of the single-layer Mxene 10a and/or the multilayer Mxene 10b. In the case of the multilayer Mxene, two adjacent Mxene layers (for example, 7a and 7b) may not necessarily be completely separated from each other, but may be partially in contact with each other.

Although not limiting the present embodiment, the thickness of each layer of Mxene (which corresponds to the Mxene layers 7a, 7b) is, for example, not less than 0.8 nm and not more than 5 nm, and particularly not less than 0.8 nm and not more than 3 nm (which can vary mainly depending on the number of M atom layers included in each layer), and the maximum dimension in a plane (two-dimensional sheet plane) parallel to the layer is, for example, not less than 0.1 μm and not more than 200 μm, and particularly not less than 1 μm and not more than 40 μm. In a case where the Mxene is the laminate (multilayer Mxene), for the individual laminate, the interlayer distance (alternatively, a void dimension indicated by Δd in FIG. 2(b)) may be, for example, 0.8 nm to 10 nm, particularly 0.8 nm to 5 nm, and more particularly about 1 nm. The multilayer Mxene that can be included is preferably Mxene having a few layers obtained through the delamination treatment. The term “the number of layers is small” means that, for example, the number of stacked layers of Mxene is 6 or less. The thickness, in a stacking direction, of the multilayer Mxene having a few layers is preferably 10 nm or less. Hereinafter, the “multilayer Mxene having a few layers” may be referred to as a “few-layer Mxene” in some cases. In addition, the single-layer Mxene and the few-layer Mxene may be collectively referred to as “single-layer/few-layer Mxene” in some cases.

The Mxene (particles) of the present embodiment preferably includes a single-layer Mxene and a few-layer Mxene, that is, a single-layer/few-layer Mxene. In the Mxene (particles), the ratio of the single-layer/few-layer Mxene having a thickness of 10 nm or less is preferably 90 vol % or more, and more preferably 95 vol % or more.

The total number of layers may be 2 or more, and may be, for example, 50 to 100,000, and particularly 1,000 to 20,000. The thickness of the conductive film in the stacking direction may be, for example, 0.1 μm to 20 μm, particularly 1 μm to 40 μm. The maximum dimension in a plane (two-dimensional sheet plane) perpendicular to the stacking direction is, for example, 0.1 μm to 100 μm, particularly 1 μm to 20 μm. Note that these dimensions can be obtained as a number average dimension (for example, a number average of at least 40) based on a photograph of a scanning electron microscope (SEM), a transmission electron microscope (TEM), or an atomic force microscope (AFM) or a distance in a real space calculated from a position on a reciprocal lattice space of a (002) plane measured by an X-ray diffraction (XRD) method.

The thickness of the conductive film containing the layered particles is preferably 0.5 μm to 20 μm. Since the impedance is stabilized and lowered by increasing the thickness of the conductive film, the thickness is preferably 0.5 μm or more. The thickness is more preferably 1.0 μm or more. The thickness is preferably as large as possible from the viewpoint of conductivity, but when flexibility or the like is required, the thickness is preferably 20 μm or less, and more preferably 15 μm or less.

The thickness of the conductive film can be measured by, for example, measurement with a micrometer, cross-sectional observation by a method such as a scanning electron microscope (SEM), a microscope, or a laser microscope.

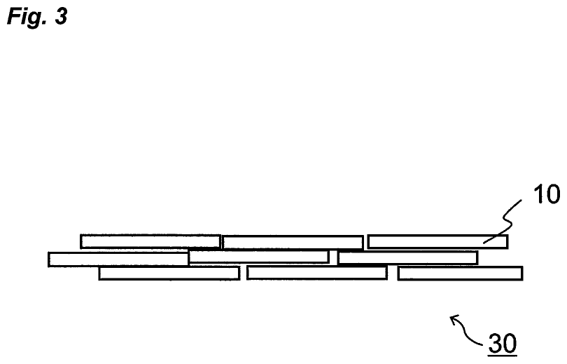

As the conductive film according to the embodiment of the present invention, for example, the conductive film 30 obtained by stacking only conductive two-dimensional particles 10 is illustrated in FIG. 3, but the present invention is not limited thereto.

The conductive film may be a conductive composite material film further containing a polymer. The polymer may be contained, for example, as an additive such as a binder added at the time of film formation, or may be added for providing strength or flexibility. In a case of the conductive composite material film, the proportion of the polymer in the conductive composite material film (when dried) may be more than 0 vol % and preferably 30 vol % or less. The proportion of the polymer may be further 10 vol % or less, and further 5 vol % or less. In other words, the proportion of the particles of the layered material in the conductive composite material film (when dried) is preferably 70 vol % or more, more preferably 90 vol % or more, and still more preferably 95 vol % or more. One electrode may be provided with a stacked film of two or more conductive composite material films having different proportions of particles of the layered material as the conductive film.

Examples of the polymer include a hydrophilic polymer having a polar group, and those in which the polar group is a group that forms a hydrogen bond with a modifier or terminal T of the layer are preferable. As the polymer, for example, one or more polymers selected from the group consisting of water-soluble polyurethane, polyvinyl alcohol, sodium alginate, an acrylic acid-based water-soluble polymer, polyacrylamide, polyaniline sulfonic acid, or nylon are preferably used.

Among these, one or more polymers selected from the group consisting of water-soluble polyurethane, polyvinyl alcohol, or sodium alginate are more preferable. As the polymer, a polymer having a urethane bond having both the hydrogen bond donor property and the hydrogen bond acceptor property is preferable, and from this viewpoint, the water-soluble polyurethane is particularly preferable.

The conductive film of the present embodiment preferably maintains a conductivity of 500 S/cm or more, for example, when the conductive film has a sheet shape and having a thickness of 5 μm. The conductivity can maintain a conductivity of preferably 1,000 S/cm or more, more preferably 1,800 S/cm or more, still more preferably 2,400 S/cm or more, and even still more preferably 2,900 S/cm or more. The conductivity of the conductive film is not particularly limited, and may be, for example, 10,000 S/cm or less. The conductivity can be determined as follows. That is, the surface resistivity is measured by a four-point probe method, a value obtained by multiplying the thickness [cm] by the surface resistivity [Ω/▭] is the volume resistivity [Ω.cm], and the conductivity [S/cm] can be obtained as the reciprocal thereof

### Porous Membrane

Next, the porous membrane will be described. The porous membrane is provided on a contact surface of the electrode with a subject, and a conductive film is formed in direct contact with a surface opposite to the contact surface. The “porous membrane” in the present specification refers to a “membrane with fine pores, which selectively permeates ions and molecules having a size smaller than the pore diameter”.

The porous membrane preferably has an average pore diameter of not less than 1 nm and not more than 1 μm. Ions derived from the subject, that is, the human body, pass through the porous membrane in contact with the subject and reach the conductive film, whereby electrodes such as myoelectric potential of the subject can be measured. That is, the porous membrane has a role of preventing direct contact between the subject and the conductive film and a role as a permeable membrane for the ions and the like. The porous membrane preferably has an average pore diameter of 1 nm or more from the viewpoint of easily transmitting the ions and the like to be carriers of current and easily reducing impedance. The average pore diameter is more preferably 10 nm or more. On the other hand, from the viewpoint of sufficiently suppressing detachment of the conductive film and exhibiting excellent performance of the conductive film over a long period of time, the average pore diameter of the porous membrane is preferably 1 μm or less, and more preferably 500 nm or less. The average pore diameter is obtained as a number average dimension (for example, a number average of at least 40 pores) by image analysis based on a photograph of a scanning electron microscope (SEM) or a transmission electron microscope (TEM).

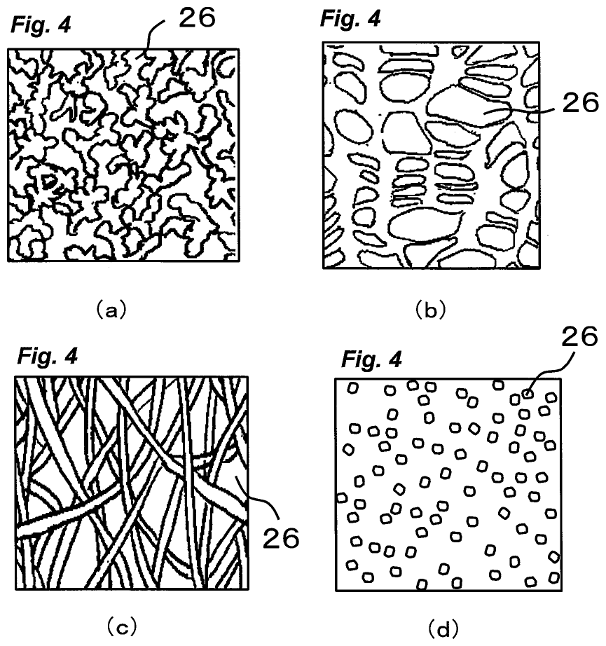

The pore shape of the porous membrane is not limited, and for example, as schematically exemplified in FIGS. 4(a) to 4(d), the porous membrane may be: 4(a) an aggregated particulate porous membrane having a plurality of pores 26, 4(b) a reticulated porous membrane, 4(c) a fibrous porous membrane, 4(d) a porous membrane having a plurality of isolated and/or communicating pipe pores (FIG. 4(d) illustrates a porous membrane in which a plurality of cylindrical holes formed perpendicular to the paper surface exist.), or a porous membrane having a honeycomb structure, which is not illustrated.

The porous membrane may be insulating or conductive. It is preferable that the porous membrane has conductivity lower than conductivity of the conductive film. It is considered that the conductivity of the conductive film is 500 S/cm or more as described above, and the porous membrane has conductivity smaller than the conductivity of the conductive film, whereby ions from the subject can be more easily moved to the conductive film, and as a result, biosignals such as myoelectric potential can be more accurately measured.

The material of the porous membrane is not particularly limited, and a porous membrane formed of an organic material, an inorganic material, or a mixture thereof can be used. Examples of the organic material having a conductivity smaller than the conductivity of the conductive film include a polymer, and examples of the inorganic material having a conductivity smaller than the conductivity of the conductive film include ceramics, or a combination thereof

The porous membrane preferably contains a hydrophilic polymer. Examples of the hydrophilic polymer include those in which a hydrophilic auxiliary agent is blended in a hydrophobic polymer to exhibit hydrophilicity, and those in which the surface of the hydrophobic polymer is subjected to a hydrophilization treatment. When the porous membrane contains the hydrophilic polymer, as described above, the adhesion to the hydrophilic conductive film (Mxene film) can be further enhanced.

As the hydrophilic polymer (which includes the polymer exhibiting hydrophilicity by mixing a hydrophilic auxiliary agent in a hydrophobic polymer, and the polymer in which a hydrophilization treatment on a surface of a hydrophobic polymer or the like is performed) capable of further enhancing adhesion with the conductive film (Mxene film), it is more preferable to contain one or more selected from the group consisting of polysulfone, cellulose acetate, regenerated cellulose, polyether sulfone, water-soluble polyurethane, polyvinyl alcohol, sodium alginate, an acrylic acid-based water-soluble polymer, polyacrylamide, polyaniline sulfonic acid, or nylon. More preferably, 50 mass % or more of the porous membrane is occupied by the hydrophilic polymer, and particularly preferably, the porous membrane is made of one or more of the hydrophilic polymers.

In addition, examples thereof include those obtained by subjecting the surface of a hydrophobic polymer (for example, olefin resins such as polyethylene and polypropylene, vinyl resins such as polystyrene and polyvinyl chloride, fluorine resins such as polyvinylidene fluoride and polytetrafluoroethylene, and polyester) to a hydrophilization treatment by various known methods such as a plasma treatment and a graft polymerization treatment. The hydrophobic polymer is more preferably one or more selected from the group consisting of polypropylene, polyethylene, polyvinylidene fluoride, or polytetrafluoroethylene. The hydrophobic polymer may be a plurality of different hydrophobic polymers, for example, polypropylene and polyethylene having a stacked structure. Instead of the hydrophobic polymer, a surface of a ceramic such as alumina, aluminum nitride, silicon nitride, or zirconium oxide may be subjected to the hydrophilization treatment.

The thickness of the porous membrane is preferably 0.1 μm to 300 μm. As the thickness of the porous membrane is thinner, ions are more likely to permeate, and impedance can be reduced. From this viewpoint, the thickness of the porous membrane is preferably 300 μm or less, more preferably 200 μm or less. On the other hand, from the viewpoint of securing durability, the thickness of the porous membrane is preferably 0.1 μm or more. The thicknesses of the porous membrane and the π-electron conjugated compound film can be measured by, for example, measurement with a micrometer, cross-sectional observation by a method such as a scanning electron microscope (SEM), a microscope, or a laser microscope.

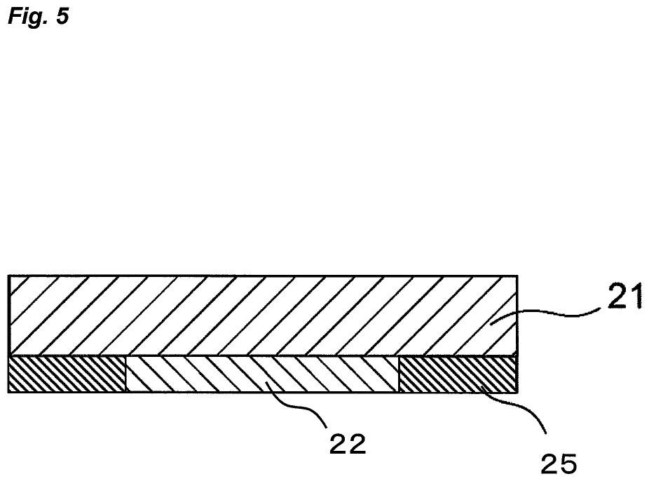

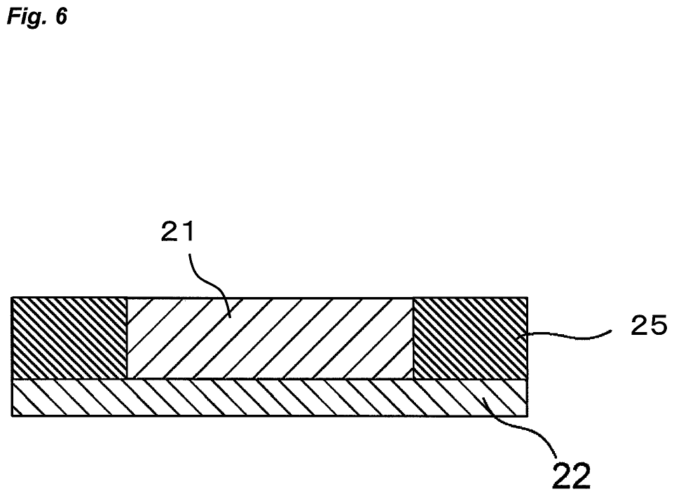

The contact area between the conductive film and the porous membrane is not particularly limited as long as the electrode exhibits high conductivity, the peeling of Mxene is suppressed, and discomfort does not occur at the time of wearing. Both the entire surfaces of the conductive film and the porous membrane may be in contact with each other, or the porous membrane may be in contact with a part of the conductive film (a), or the conductive film (b) may be in contact with a part of the porous membrane. FIG. 5 is a diagram illustrating an example of the above (a), and a porous membrane 22 and, for example, an insulating film 25 are provided on a surface of the conductive film 21 on the subject side. FIG. 6 is a diagram illustrating an example of the above (b), and a porous membrane 22 is provided on a surface formed of a conductive film 21 and, for example, an insulating film 25. From the viewpoint of more easily achieving the above characteristics, the contact area ratio of the conductive film with the porous membrane on the surface on the subject side is preferably 60% or more, more preferably 80% or more, and most preferably 100%.

As the porous membrane, a commercially available product may be used, and the porous membrane may be obtained by a method such as a phase conversion method, a melt quenching method, an extraction method, or an electron beam irradiation method. The phase conversion method is a method in which micropores are formed by utilizing a two-phase separation phenomenon that occurs when a film-forming solution (cast solution) prepared by dissolving an organic polymer in an organic solvent is cast on a glass plate or the like, and then the glass plate or the like is immersed in an appropriate gelling solution (insoluble organic solvent of organic polymer, water, and the like) or dried. The melt quenching method is a method in which a film is formed using a varnish in which a solvent and a polymer are combined so that a large difference in solubility occurs depending on a temperature, and then the film is quenched and solidified. The extraction method (replica method) is a method in which an additive that can be easily extracted in a subsequent step is added to a polymer solution or a dispersion liquid, the additive is formed into a film, and then the additive is extracted by an appropriate method. The electron beam irradiation method is a method in which a polymer thin film having a thickness of about 10 μm is irradiated with an electron beam (charged particles) to form a particle trajectory on the film, and then performing an etching treatment with a solvent to widen the trajectory to form fine pores.

### Biosignal Sensing Electrode

The biosignal sensing electrode of the present embodiment includes a stacked layer in which a conductive film and a porous membrane are in direct contact with each other, and is not limited to a specific form as long as the porous membrane is provided on a contact surface with the subject. Examples of the electrode include an electrode in a solid state and an electrode in a flexible and soft state. However, it is preferable to have flexibility as much as possible from the viewpoint of followability to a living body (skin), suppression of electrode breakage, and the like.

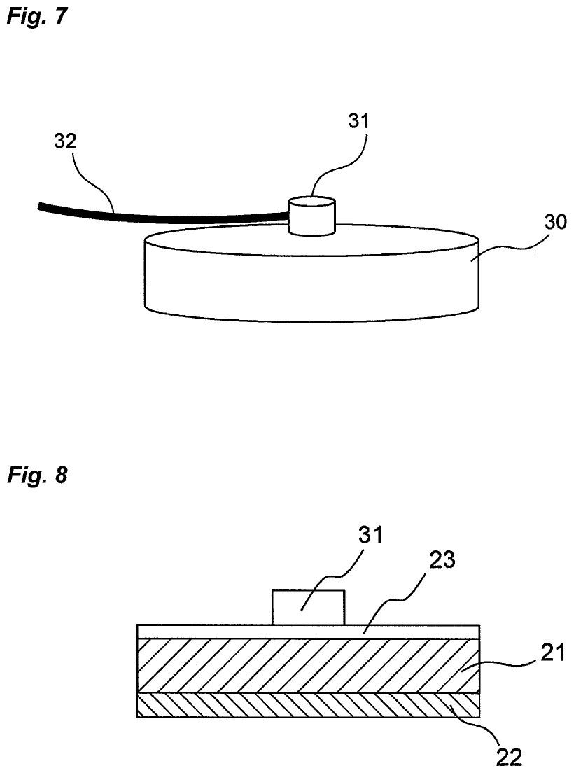

As an embodiment of the biosignal sensing electrode, FIG. 7 illustrates a schematic perspective view of a snap-type electrode. FIG. 7 illustrates a schematic perspective view of a snap-type electrode in which a lead wire 32 is connected to a snap portion 31 of an electrode 30 whose contact surface with the subject is a flat surface. An example of a cross-sectional view of the electrode 30 of FIG. 7 is schematically illustrated in FIG. 8.

In FIG. 8, the conductive film 21 is formed on a substrate 23 formed of a conductive material. In this manner, since the conductive film 21 is formed and the porous membrane 22 is formed as a contact surface with the subject, it is possible to provide a biosignal sensing electrode having high sensitivity and reduced discomfort of wearing.

Examples of the conductive material constituting the substrate 23 include at least one material of metal materials such as gold, silver, copper, platinum, nickel, titanium, tin, iron, zinc, magnesium, aluminum, tungsten, and molybdenum, and a conductive polymer.

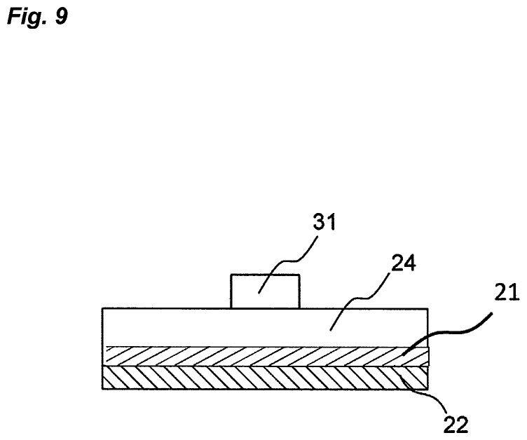

As another embodiment, as illustrated in FIG. 9, the substrate may be a conventional snap-type electrode 24. As the conductive material constituting the snap-type electrode, the same material as the substrate 23 formed of the conductive material can be used. According to the above configuration, since the extraction electrode having versatility is used, it is possible to provide a biosignal sensing electrode with low cost and high sensitivity.

In addition, as another embodiment, when the conductive film is a conductive composite material film of a Mxene film and a polymer, an electrode which is a stacked film of a conductive composite material film and a porous membrane and does not have a substrate can be used.

Since the biosignal sensing electrode of the present embodiment does not contain moisture as in Patent Document 1, it does not feel uncomfortable like being wet at the time of wearing. In addition, when moisture is contained as in Patent Document 1, an impedance change due to drying may occur. On the other hand, since the biosignal sensing electrode of the present embodiment is a dry electrode, there is no impedance change due to the drying, and the signal reliability is high.

Since the biosignal sensing electrode of the present embodiment includes a low-impedance Mxene film as an electric conductor, signal accuracy is high. Furthermore, since stacked layer of the conductive film (Mxene film) and the porous membrane is flexible, it is not necessary to provide a layer for skin followability. Therefore, in the biosignal sensing electrode of the present embodiment, the number of layers is small, and low impedance can be more easily realized. On the other hand, for example, in Patent Document 2, the electric conductor is hard, and a layer of a conductive medium is essential from the viewpoint of skin followability, and as a result, the number of layers is large and the impedance is high.

In addition, in the biosignal sensing electrode of the present embodiment, since the conductive film is protected by the porous membrane, and the contact layer in contact with the subject is the porous membrane, Mxene can be prevented from being detached from the conductive film. The porous membrane has ion conductivity through which ions from a subject easily permeate, and has low impedance.

When the conductive film and the porous membrane are in direct contact with each other, the adhesion between the conductive film (Mxene film) exhibiting hydrophilicity and the porous membrane preferably containing a hydrophilic polymer is enhanced, and high adhesion can be secured without newly providing an intermediate layer containing a pressure-sensitive adhesive or the like between the conductive film and the porous membrane. As a result, the number of layers is small, the movement distance of ions from the subject to the conductive film through the porous membrane is shortened, low impedance is more easily realized, and the sensitivity of the electrode can be further enhanced.

### Method for Producing Biosignal Sensing Electrode

A method for producing an electrode of the present embodiment using Mxene produced as described above is not particularly limited. When the conductive film sheet of the present embodiment has a sheet-like form, for example, as illustrated below, an electrode can be formed.

First, a Mxene aqueous dispersion or a Mxene organic solvent dispersion in which the Mxene particles (particles of a layered material) are prepared. The solvent of the Mxene aqueous dispersion is typically water, and in some cases, other liquid substances may be contained in a relatively small amount (for example, 30 mass % or less, preferably 20 mass % or less based on the whole mass) in addition to water.

More specifically, as described above, the operation of subjecting the Mxene-containing aqueous mixture obtained by selectively etching the A atom from the MAX phase to solid-liquid separation (for example, sedimentation, centrifugation, and the like), partially removing the aqueous solvent (liquid phase) from the mixture, adding a fresh aqueous solvent to the mixture, and applying shear force to the mixture may be performed at any appropriate timing to obtain a Mxene-containing aqueous medium. Such an operation may be performed once, or may be repeated twice or more in some cases.

Before drying, a precursor of the conductive film (also referred to as a “precursor film”) may be formed using an Mxene-containing aqueous medium such as an Mxene aqueous dispersion or an Mxene organic solvent dispersion. The method for forming the precursor film is not particularly limited, and for example, coating, suction filtration, spray, or the like can be used.

More specifically, the Mxene-containing aqueous medium is applied to the substrate as it is or after being appropriately adjusted (for example, dilution with an aqueous solvent or addition of a binder). Examples of the coating method include a spray coating method in which spray coating is performed using a nozzle such as a one-fluid nozzle, a two-fluid nozzle, or an air brush, a slit coating method using a table coater, a comma coater, or a bar coater, a screen printing method, a metal mask printing method, a spin coating, dip coating, or dropping. A substrate formed of a metal material, a resin, or the like suitable for the biosignal sensing electrode can be appropriately adopted as the substrate. By coating onto any suitable substrate (which may constitute a predetermined member together with the conductive film, or may be finally separated from the conductive film), a precursor film can be formed on the substrate.

In addition, the Mxene-containing aqueous medium is appropriately adjusted (for example, diluted with an aqueous medium), and is subjected to suction filtration through a filter (which may constitute a predetermined member together with the conductive film, or may be finally separated from the conductive film) installed in a nutsche or the like. Thereby, the aqueous medium is at least partially removed, so that a precursor can be formed on the filter. The filter is not particularly limited, but a membrane filter or the like can be used. By performing the suction filtration, a conductive film can be produced without using the binder or the like.

Next, the precursor formed as described above is dried to obtain, a conductive film 30 as schematically illustrated in FIG. 3. In the present invention, the “drying” means removing the aqueous solvent that can exist in the precursor.

Drying may be performed under mild conditions such as natural drying (typically, it is disposed in an air atmosphere at normal temperature and normal pressure.) or air drying (blowing air), or may be performed under relatively active conditions such as hot air drying (blowing heated air), heat drying, and/or vacuum drying. The drying may be performed, for example, at a temperature of 400° C. or lower using a normal pressure oven or a vacuum oven.

The forming and drying the precursor may be appropriately repeated until a desired conductive film thickness is obtained. For example, a combination of spraying and drying may be repeated a plurality of times.

Even in a case where the conductive film contains a polymer, an electrode including the conductive composite material is not particularly limited. When the conductive composite material of the present embodiment has a sheet-like form, for example, as illustrated below, the layered material and the polymer can be mixed to form a coating film.

First, a Mxene aqueous dispersion, a Mxene organic solvent dispersion, or a Mxene powder in which the Mxene particles (particles of a layered material) are present in a solvent may be mixed with a polymer. The solvent of the Mxene aqueous dispersion is typically water, and in some cases, other liquid substances may be contained in a relatively small amount (for example, 30 mass % or less, preferably 20 mass % or less based on the whole mass) in addition to water.

The stirring of the Mxene particles and the polymer can be performed using a dispersing device such as a homogenizer, a propeller stirrer, a thin film swirling stirrer, a planetary mixer, a mechanical shaker, or a vortex mixer.

A slurry which is a mixture of the Mxene particles and the polymer may be applied to a substrate (for example, a substrate), but the application method is not limited. Examples of the coating method include a spray coating method in which spray coating is performed using a nozzle such as a one-fluid nozzle, a two-fluid nozzle, or an air brush, a slit coating method using a table coater, a comma coater, or a bar coater, a screen printing method, a metal mask printing method, a spin coating, dip coating, or dropping. As described above, a substrate formed of a metal material, a resin, or the like suitable for the biosignal sensing electrode can be appropriately adopted as the substrate.

The coating and drying may be repeated a plurality of times as necessary until a film having a desired thickness is obtained. The drying and curing may be performed, for example, at a temperature of 400° C. or lower using a normal pressure oven or a vacuum oven.

After the Mxene film is formed by any of the methods described above, before the Mxene film is dried and cured, for example, a commercially available product is stacked as a porous membrane as described above, and then the Mxene film is dried and cured, or after the Mxene film is dried and cured, a porous membrane may be formed on the surface of the Mxene film by the above-described phase conversion method or the like.

Although the biosignal sensing electrode in one embodiment of the present invention has been described in detail above, various modifications are possible. It should be noted that the biosignal sensing electrode of the present invention may be produced by a method different from the producing method in the above-described embodiment.

### EXAMPLES

### Preparation of MAX Particles

TiC powder, Ti powder, and Al powder (all manufactured by Kojundo Chemical Laboratory Co., Ltd.) were placed in a ball mill containing zirconia balls at a molar ratio of 2: 1: 1 and mixed for 24 hours. The obtained mixed powder was fired at 1350° C. for 2 hours under an Ar atmosphere. The fired body (block-shaped MAX) thus obtained was pulverized with an end mill to a maximum dimension of 40 μm or less. In this way, Ti₃AlC₂ particles were obtained as MAX particles.

### Preparation of Mxene Dispersion

1 g of the Ti₃AlC₂ particles (powder) prepared above was weighed, added to 10 Ml of 9 mol/L hydrochloric acid together with 1 g of LiF using a fluororesin container, stirred with a stirrer at 35° C. for 24 hours, and a solid-liquid mixture (suspension) containing a solid component derived from the Ti₃AlC₂ powder was obtained. The solid-liquid mixture (suspension) having been etched was transferred to a centrifuge tube, pure water was added thereto, the mixture was stirred, a supernatant and a precipitate were separated with a centrifuge, and the supernatant was discarded. This was repeated 10 times for washing. Thereafter, a delamination treatment was performed by performing a treatment for a predetermined time using a mechanical shaker. Thereafter, the supernatant was recovered by centrifugation, and the supernatant was used as a Mxene aqueous dispersion.

### Preparation of Biosignal Sensing Electrode Sample

A hydrophilic porous membrane (Product number GPWP04700, hydrophilic polyethersulfone (PES) membrane, manufactured by Merck KgaA, thickness: about 175 μm, pore diameter: 0.22 μm) was cut with scissors so as to have an area of 196 mm², and an aqueous dispersion containing 4.5 mass % of Mxene was sprayed thereon for 3 seconds, and then temporarily dried with a dryer. This spraying and temporary drying were repeated five times, and then finally dried at 80° C. for 30 minutes in an oven, and a stacked film of a conductive film (Mxene film) having a thickness of 5 μm and the hydrophilic porous membrane was used as a biosignal sensing electrode sample. As Comparative Example 1, a biosignal sensing electrode sample prepared in the same manner as described above except that the porous membrane was not provided and only the Mxene film (Mxene film having an area of 196 mm² and a thickness of 5 μm) was provided was also prepared. As Comparative Example 2, a monitoring electrode (product number 2228) manufactured by 3M Company, which is a commercially available bioelectrode, was also prepared.

The impedance of the biosignal sensing electrode sample (Mxene film+porous membrane) according to the present embodiment, the impedance of the biosignal sensing electrode sample (only Mxene film) according to Comparative Example 1, and the impedance of the commercially available electrode according to Comparative Example 2 were measured by the following methods.

Each of the above electrodes (samples) was brought into contact with a chicken skin removed, which is considered to be equivalent to human skin, and the impedance was measured with an impedance measuring device Autolab (manufactured by Metrohm Autolab). As measurement conditions, a measurement frequency was set to 1 Hz, 10 Hz, or 1,000 Hz, and an effective voltage was set to 10 Mv.

As a measurement level, there are three levels in total as follows:

Level of biosignal sensing electrode according to the present embodiment produced above for both working electrode and counter electrode,

Level of biosignal sensing electrode (only the Mxene film) according to Comparative Example 1 for both working electrode and counter electrode, and

Level of commercially available bioelectrode according to Comparative Example 2 for both working electrode and counter electrode. Since the measurement was performed with two poles, the reference electrode was not used. The measurement results are shown in Table 1 below.

TABLE 1

| | Impedance | Impedance | Impedance |
|---|---|---|---|
| | \|Z\| (Ω) | \|Z\| (Ω) | \|Z\| (Ω) |
| | at 1 Hz | at 10 Hz | at 1000 Hz |
| Present Embodiment | 272.4 | 196.5 | 159.6 |
| Mxene film only | 201.6 | 161.4 | 110.7 |
| Commercially | 343.2 | 231.5 | 208.5 |
| available electrode | | | |

From the results in Table 1 above, it has been found that the biosignal sensing electrode sample (Mxene film+porous membrane) according to the present embodiment has the same impedance as the impedance of the Mxene film alone at any frequency, and the increase in impedance is small even when the porous membrane is formed. In addition, it can be seen that the biosignal sensing electrode sample of the present invention has a lower impedance than a commercially available Ag/AgCl gel electrode, and has sufficiently low resistance as a biological signal electrode. Further, in the biosignal sensing electrode of the present invention, since the Mxene film and the porous membrane are in direct contact with each other and have good adhesion, it is not necessary to provide an adhesive layer between the MXene film and the porous membrane, and as a result, the number of layers is reduced, and a low and stable impedance is exhibited. Furthermore, according to the present embodiment, since the Mxene film does not come into contact with the subject, peeling of Mxene is suppressed, and measurement can be stably performed for a long period of time. Furthermore, since it does not retain moisture or the like, discomfort of wearing can be reduced.

The biosignal sensing electrode of the present invention can be preferably used for a device that extracts and measures a biosignal such as an electromyogram signal or an electrocardiogram signal.

### DESCRIPTION OF REFERENCE SIGNS

1a, 1b: Layer body (MₘXₙ layer)

3a, 5a, 3b, 5b: Modifier or terminal T

7a, 7b: MXene layer

10, 10a, 10b: MXene (layered material)

21: Conductive film

22: Porous membrane

23: Substrate formed of conductive material

24: Conventional snap-type electrode

25: Insulating film

26: Hole

30: Biosignal sensing electrode

31: Electrode snap portion

32: Lead wire

1. A biosignal sensing electrode comprising:
a conductive film containing particles of a layered material including one or plural layers, wherein the one or plural layers includes a layer body represented by:
MₘXₙ
wherein M is at least one metal of Group 3, 4, 5, 6, or 7,
X is a carbon atom, a nitrogen atom, or a combination thereof,
n is 1 to 4,
m is more than n and 5 or less, and
a modifier or terminal T exists on a surface of the layer body, wherein T is at least one selected from the group consisting of a hydroxyl group, a fluorine atom, a chlorine atom, an oxygen atom, or a hydrogen atom; and
a porous membrane that contains a hydrophilic polymer, the porous membrane having a first surface opposing at least part of the conductive film and a second surface defining a contact surface with a subject.

2. The biosignal sensing electrode according to claim 1, wherein the conductive film contains a polymer.

3. The biosignal sensing electrode according to claim 1, wherein the porous membrane has an average pore diameter of 1 nm to 1 μm.

4. The biosignal sensing electrode according to claim 1, wherein a thickness of the porous membrane is 0.1 μm to 300 μm.

5. The biosignal sensing electrode according to claim 1, wherein a thickness of the conductive film is 0.5 μm to 20 μm.

6. The biosignal sensing electrode according to claim 1, wherein the porous membrane is an aggregated particulate porous membrane having a plurality of pores.

7. The biosignal sensing electrode according to claim 1, wherein the porous membrane is a reticulated porous membrane.

8. The biosignal sensing electrode according to claim 1, wherein the porous membrane is a fibrous porous membrane.

9. The biosignal sensing electrode according to claim 1, wherein the porous membrane defines a plurality of isolated and/or communicating pipe pores.

10. The biosignal sensing electrode according to claim 1, wherein the porous membrane has a conductivity lower than a conductivity of the conductive film.

11. The biosignal sensing electrode according to claim 1, wherein the hydrophilic polymer is one or more selected from the group consisting of polysulfone, cellulose acetate, regenerated cellulose, polyether sulfone, water-soluble polyurethane, polyvinyl alcohol, sodium alginate, an acrylic acid-based water-soluble polymer, polyacrylamide, polyaniline sulfonic acid, nylon, olefin resins, vinyl resins, fluorine resins, and polyester.

12. The biosignal sensing electrode according to claim 1, wherein the hydrophilic polymer is 50 mass % or more of the porous membrane.

13. The biosignal sensing electrode according to claim 1, wherein a contact area ratio of the conductive film with the porous membrane is 60% or more.

14. The biosignal sensing electrode according to claim 1, wherein the conductive film and the porous membrane are in direct contact with each other.

15. The biosignal sensing electrode according to claim 1, further comprising a conductive substrate supporting the conductive film on a side thereof opposite to the porous membrane.
